(19) United States
(12) Patent Application Publication
(10) Pub. No.: US 2023/0114795 A1
(43) Pub. Date: Apr. 13, 2023

(54) STRAINS CAPABLE OF REDUCING HEAVY METAL CONTENTS IN VEGETABLES AND IMPROVING QUALITY OF VEGETABLES AND APPLICATION THEREOF

(71) Applicant: QILU UNIVERSITY OF TECHNOLOGY, Jinan (CN)

(72) Inventors: Tao XIA, Jinan (CN); Xiaohan WANG, Jinan (CN)

(21) Appl. No.: 18/060,251

(22) Filed: Nov. 30, 2022

Related U.S. Application Data
(63) Continuation of application No. PCT/CN2021/082435, filed on Mar. 23, 2021.

(30) Foreign Application Priority Data
Jun. 11, 2020 (CN) 202010531482.9

(51) Int. Cl. C12N 1/20; A01G 22/15

(52) U.S. Cl. CPC C12N 1/20; A01G 22/15

(57) ABSTRACT

Disclosed are strains capable of reducing heavy metal contents in vegetables and improving quality of vegetables and application thereof. Specifically, the strains are strains W7 and W25, which can reduce heavy metal contents, especially cadmium (Cd) contents, in plants, promote growth of plants, increase Vc and soluble protein contents in plants, and increase a microbial abundance and a urease activity in the soil at the same time, and can be widely applied to the fields of soil improvement, crop biomass increase, and soil heavy metal restoration.

### CROSS REFERENCES

This application is the U.S. continuation application of International Application No. PCT/CN2021/082435 filed on 23 Mar. 2021 which designated the U.S. and claims priority to Chinese Application Nos. filed CN 202010531482.9 filed on 11 Jun. 2020, the entire contents of each of which are hereby incorporated by reference.

### FIELD OF THE INVENTION

The present disclosure belongs to the technical field of agricultural microbe application, and specifically relates to strains capable of reducing heavy metal contents in vegetables and improving quality of vegetables and application thereof.

### BACKGROUND OF THE INVENTION

Cadmium (Cd) is one of the most toxic heavy metals, which is extremely harmful to human health. Farmland soil cadmium contamination caused by the excessive use of chemical fertilizers and pesticides, sewage irrigation, and mining activities has become a concern of all countries in the world. Cd contamination is the most serious heavy metal contamination in China, accounting for about 7% of farmland contamination. Cadmium is accumulated in vegetables, crops, fruit trees, forage grasses, and other plants growing in the mildly and moderately cadmium-contaminated soil through food chains, causing hazards to human and animal health.

In situ immobilization of Cd is an effective measure to reduce the Cd transfer from the soil and ensure the food safety. Soil heavy metal fixing agents reduce the bioavailability of Cd and reduce the transfer of Cd from the soil to plants through adsorption, complexation, and precipitation. However, continuous addition of these organic or inorganic fixing agents may be harmful to properties, the structure, and the ecosystem of the soil.

Chinese patent document CN110846250A (Application No. 201911142617.6) discloses high-yield γ-PGA-producing Bacillus subtilis and application thereof. According to this disclosure, the combination of Bacillus subtilis and biochar can effectively promote the growth of plants and reduce the absorption of the heavy metal Pb by the plants from the soil.

There are many related reports on application of microbial strains in degradation of heavy metals and promotion of the growth of plants in the prior art, but in most of them, it is necessary to combine multiple microbes or combine a microbe with other substances to achieve multiple effects. However, there are very few reports on application of single multifunctional strains on crop planting and soil improvement. Therefore, the development of multifunctional microbial strains is also an urgent problem to be solved in the technical field of agricultural microbes currently.

### SUMMARY OF THE INVENTION

In view of the deficiencies in the prior art, the present disclosure provides two strains capable of reducing heavy metal contents in vegetables and improving quality of vegetables and application thereof.

The strains of the present disclosure can reduce heavy metal contents, especially cadmium (Cd) contents, in plants, promote growth of plants, increase Vitamin C (Vc) and soluble protein contents in plants, and increase a microbial abundance and a urease activity in the soil at the same time. Using plant growth promoting bacteria is a safe, ecological, economical, environmentally-friendly, and effective way to reduce heavy metal contents in plants, improve the activity of soil, reduce the transfer of heavy metals to plants, and ensure the safety of agricultural products.

A Bacillus strain W7, taxonomically named Bacillus subtilis W7, was deposited in China General Microbiological Culture Collection Center (CGMCC) at No. 3, Yard 1, Beichen West Road, Chaoyang District, Beijing, China on Jun. 11, 2020, with the number of CGMCC20043, that was obtained by mutagenesis with ethyl methanesulfonate (EMS).

A culture method of the strain W7 includes the following the steps:

inoculating the strain W7 onto a solid medium and culturing same at 28 to 32° C. for 2 to 3 d, picking colonies, inoculating same into a liquid medium, and culturing same at 35 to 37° C. and 150 to 200 rpm for 16 to 20 h to obtain a fermentation broth of the strain W7. The solid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, 20 g/L of agar, and the balance of water; and the liquid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, and the balance of water.

The strain W7 is applied to preparation of a biological agent.

According to the present disclosure, preferably, the biological agent is a liquid agent containing the strain W7 at a concentration of more than 1×10⁸ CFU/mL.

The strain W7 is applied to crop planting.

According to the present disclosure, preferably, the strain W7 is used to reduce a heavy metal cadmium content in a vegetable.

According to the present disclosure, preferably, the strain W7 is used to increase Vitamin C and soluble protein contents in the vegetable.

Further preferably, the vegetable is lettuce.

The strain is applied to soil improvement.

According to the present disclosure, preferably, the strain W7 is used to fix the heavy metal cadmium in the soil.

According to the present disclosure, preferably, the strain W7 is used to promote proliferation of microbes in the soil.

Further preferably, the strain W7 is used to promote proliferation of one or more of Proteobacteria, Firmicutes, Sphingomonas, and Aneurinibacillus in the heavy metal cadmium-contaminated soil.

A Bacillus strain W25, taxonomically named Bacillus amyloliquefaciens, was deposited in China General Microbiological Culture Collection Center (CGMCC) at No. 3, Yard 1, Beichen West Road, Chaoyang District, Beijing, China on Jun. 11, 2020, with the number of CGMCC20042, that was obtained by mutagenesis with ethyl methanesulfonate (EMS).

A culture method of the strain W25 includes the following steps:

inoculating the strain W25 onto a solid medium and culturing same at 28 to 32° C. for 2 to 3 d, picking colonies, inoculating same into a liquid medium, and culturing same at 35 to 37° C. and 150 to 200 rpm for 16 to 20 h to obtain a fermentation broth of the strain W25. The solid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, 20 g/L of agar, and the balance of water; and the liquid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, and the balance of water.

The strain W25 is applied to preparation of a biological agent.

According to the present disclosure, preferably, the biological agent is a liquid agent containing the strain W25 at a concentration of 1×10⁸ CFU/mL.

The strain W25 is applied to crop planting.

According to the present disclosure, preferably, the strain W25 is used to reduce a heavy metal cadmium content in a vegetable.

According to the present disclosure, preferably, the strain W25 is used to increase Vitamin C and soluble protein contents in the vegetable.

Further preferably, the vegetable is lettuce.

The strain W25 is applied to soil improvement.

According to the present disclosure, preferably, the strain W25 is used to fix the heavy metal cadmium in the soil.

According to the present disclosure, preferably, the strain W25 is used to promote proliferation of microbes in the soil.

Further preferably, the strain W25 is used to promote proliferation of one or more of Proteobacteria, Firmicutes, Sphingomonas, and Aneurinibacillus in the heavy metal cadmium-contaminated soil.

### Beneficial Effects of the Invention

1. The present disclosure provides two strains W7 and W25 capable of achieving multiple effects at the same time.
2. The strains of the present disclosure can reduce heavy metal contents, especially cadmium contents, in plants, promote growth of plants, increase Vc and soluble protein contents in plants, and increase a microbial abundance and a urease activity in the soil at the same time.
3. The strains of the present disclosure are capable of producing γ-PGA.

### BRIEF DESCRIPTION OF THE DRAWINGS

FIG. 1 is a diagram showing a determination result of Cd contents in an edible tissue and a root of lettuce in a pot experiment of Example 3;

FIG. 2 is a diagram showing a determination result of γ-PGA contents corresponding to different culture time of a culture solution of Example 4;

FIG. 3 is a diagram showing a determination result of Cd contents corresponding to different culture time of a culture solution with an initial Cd content of 3 mg·L⁻¹ of Example 4;

FIG. 4 is a diagram showing a determination result of Cd contents corresponding to different culture time of a culture solution with an initial Cd content of 6 mg·L⁻¹ of Example 4;

FIG. 5 is a diagram showing a determination result of OD₆₀₀ values corresponding to different culture time of a culture solution of Example 4;

FIG. 6 is a diagram showing a determination result of pH values corresponding to different culture time of a culture solution of Example 4;

FIG. 7 is a diagram showing a determination result of a urease activity in collected soil of Example 5;

FIG. 8 is a diagram showing a detection result of Proteobacteria in the collected soil of Example 5;

FIG. 9 is a diagram showing a detection result of Firmicutes in the collected soil of Example 5;

FIG. 10 is a diagram showing a detection result of Sphingomonas in the collected soil of Example 5; and

FIG. 11 is a diagram showing a detection result of Bacillus in the collected soil of Example 5.

### DETAILED DESCRIPTION OF THE EMBODIMENTS

The present disclosure will be further described below with reference to specific examples, but the scope of protection is not limited thereto.

Unless other defined, the following examples are implemented according to conventional experimental methods and operation steps in the art.

Preservation Information of Biological Samples

A Bacillus strain W7, taxonomically named Bacillus subtilis W7, was deposited in China General Microbiological Culture Collection Center (CGMCC) at No. 3, Yard 1, Beichen West Road, Chaoyang District, Beijing, China on Jun. 11, 2020, with the number of CGMCC20043.

A Bacillus strain W25, taxonomically named Bacillus amyloliquefaciens, was deposited in China General Microbiological Culture Collection Center (CGMCC) at No. 3, Yard 1, Beichen West Road, Chaoyang District, Beijing, China on Jun. 11, 2020, with the number of CGMCC20042.

### Example 1

Screening of the Strains W7 and W25

Mutagenesis

Bacillus subtilis or Bacillus amyloliquefaciens was treated 0.5% ethyl methanesulfonate (EMS) for 45 min.

Primary Screening

Screening was carried out by streaking samples on isolation plates containing neutral red (Sigma-Aldrich, USA). The isolation medium composed of 1% glucose, 0.5% yeast extract, 0.5% L-glutamate, 0.05% KH2PO4, 0.05% K2HPO4, 0.01%, MgSO47H2O, and 1.5% agar. The medium without agar was also used as seed medium. Typically, 0.006% (w/v) neutral red was added into the isolation medium for primary screening. Initial pH of the medium was adjusted to 7.2±0.1 by using 2 N NaOH and/or 2 N HCl. The medium was autoclaved for 20 min at 121° C. After incubation at 37° C., any colony which formed a specific concentric zone by interacting with the dye was considered as a potent γ-PGA-producing strain and selected for further analysis.

Culture of Isolates

Single, rapid growing colonies with clear concentric zone were picked from the plates, inoculated into 50 ml sterile tubes containing 5 ml basic fermentation medium (3% glucose, 0.25% yeast extract, 2% L-glutamate, 0.05% KH2PO4, 0.05% K2HPO4, 0.01% MgSO4.7H2O, pH7.2±0.1) and cultured at 37° C. with shaking at 200 rpm for 24 h.

Purification and Detection of γ-PGA

The fermentation culture was appropriately diluted with distilled water, and cells were separated by centrifugation for 20 min at 12,000 rpm. The supernatant was gently mixed with four volumes of ice-cold ethanol. After centrifugation, the sediment was collected, dissolved in appropriate volume of deionized water, and then centrifuged to remove any insoluble contaminants. The solution containing 7-PGA was used for quantitative analysis using the UV spectrophotometric method. The absorbance of all samples and controls at 216 nm against deionized water was measured by a UV/vis microplate spectrophotometer. A standard curve was generated by plotting the average blank corrected absorbance of each standard at 216 nm versus its concentration in micrograms per milliliter (20-200 g/ml). Wild-type Bacillus subtilis and Bacillus subtilis W7 produces 3.8 and 6.4 g/L of γ-PGA, respectively, while Wild-type Bacillus amyloliquefaciens and Bacillus amyloliquefaciens W25 produces 5.4 and 8.5 g/L of γ-PGA, respectively.

Soil samples were collected from the rhizosphere soil of asparagus lettuce growing in a Cd-contaminated farmland (with a Cd content in the soil of 1.35 mg·kg⁻¹) in a suburb of Jinan. 10 g of sample was suspended in 90 mL of sterile distilled water and boiled for 5 min, and the diluted suspension was smeared onto an isolation plate. An isolation medium contains 10 g/L of glucose, 5 g/L of yeast extract, 5 g/L of L-sodium glutamate, 0.5 g/L of KH₂PO₄, 0.5 g/L of K₂HPO₄, 0.1 g/L of MgSO₄.7H₂O, 0.06 g/L of neutral red, 15 g/L of agar, and the balance of water. An initial pH value of the medium was regulated to 7.2±0.1. The suspension was incubated in the dark at 37° C. for 48 h and then reacted with the dye (the color changed from red to yellow) to form colonies in a specific concentric zone, which were γ-PGA-producing strains, and the γ-PGA-producing strains obtained by screening were numbered W7 and W25.

### Example 2

Respective Activation and Preparation of Bacterial Suspensions of the Strains W7 and W25

Slant W7 bacteria were inoculated onto a solid medium and cultured at 30° C. for 3 d. Then, full and thick W7 colonies were selected, inoculated into a liquid medium, and subjected to shake culture at 37° C. and 150 rpm for 20 h. The fermentation broth was transferred into a sterile centrifugal bottle and centrifuged at 5,000 rpm for 5 min, bacteria were collected, washed with sterile deionized water, and resuspended to make the concentration greater than 5×10⁸ CFU/mL. The solid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, 20 g/L of agar, and the balance of water; and the liquid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, and the balance of water.

Activation and preparation of a bacterial suspension of the strain W25 were the same as those of the strain W7.

### Example 3

Respective Application of the Strains W7 and W25 in Crop Planting

W7 was applied to a potted lettuce experiment according to the following methods: each pot had a diameter of 28 cm and a height of 35 cm and contained 4.8 kg of soil, additional cadmium (CdCl₂.2.5H₂O) was added until final Cd concentrations in the soil were 0, 0.5, and 1 mg/kg, respectively, the additional cadmium was mixed with the soil thoroughly, and the soil was balanced for 45 d; and 3 parallels were prepared for each concentration group. Harvested lettuce seeds subjected to surface sterilization were sowed into each pot, after sprouting, the plants were thinned out according to 15 plants/pot; 3 parallels were prepared for each concentration group; the soil was watered regularly to keep it moist, before inoculation, the bacteria prepared in Example 2 were selected and diluted with sterile deionized water to 1×10⁸ CFU/mL. Sterile deionized water was taken as a control, at the third true-leaf stage of lettuce, a groove (1 to 2 cm in depth) was dug around a root of lettuce, the bacterial suspension was added into the groove according to an additive volume of 90 mL/pot; and the potted plants grew in a greenhouse (with the temperature of 10 to 22° C., the relative humidity of 30 to 45%, and normal light) for 45 d.

After growth, an edible tissue and a root of each potted lettuce were collected for the following analysis; the edible tissue and the root of lettuce were washed with 0.01 M ethylene diamine tetraacetic acid (EDTA) and distilled water and then divided into two parts equally. One part was inactivated at 105° C. for 30 min and dried to constant weight at 65° C., and the weight after drying was recorded; the dried edible tissue and root were milled and digested for determination of a Cd content. Vitamin C (Vc) and soluble protein contents in the fresh edible tissue were determined by a standard method.

A pot experiment of the strain W25 was implemented by a method the same as that of the pot experiment of the strain W7.

A method for determining the Cd content: 0.1 g of plant sample was weighed accurately, digested by means of microwave, and added with 5% HNO3 to constant volume, and a Cd content in the digestion solution was determined by using ICP-OES with reference to the method disclosed by Gao Yuanyuan, Peng Zhaofeng, Qiu Haiou, Gu Yansheng, and Cheng Wei in Determination of heavy metal elements in dominant plants from Hubei Zigui Yueliangbao gold mine tailings with ICP-OES [J], Chinese Journal of Analysis Laboratory, 2016, 35(5): 521-525.

A method for determining the Vitamin C content: the Vitamin C content in the edible tissue of the vegetable was determined with reference to the method disclosed by Zhao Xiaomei, Jiang Ying, Wu Yupeng, Liu Kuan, and Zhang Zhiqiang in Assay research on VC content in fruit and vegetable [J], Food Science, 2006, 27(3): 197-199.

A method for determining the soluble protein content: the soluble protein content in the edible tissue of the vegetable was determined with reference to the method disclosed by Zhao Yingyong, Dai Yun, Cui Xiuming, Zhang Wenbin, and Ma Ni in Determination of protein contents of radix aconiti kusnezoffii using Coomassie brilliant blue G-250 dye binding [J], Journal of Yunan Minzu University: Natural Sciences Edition, 2006, 15(3): 235-237.

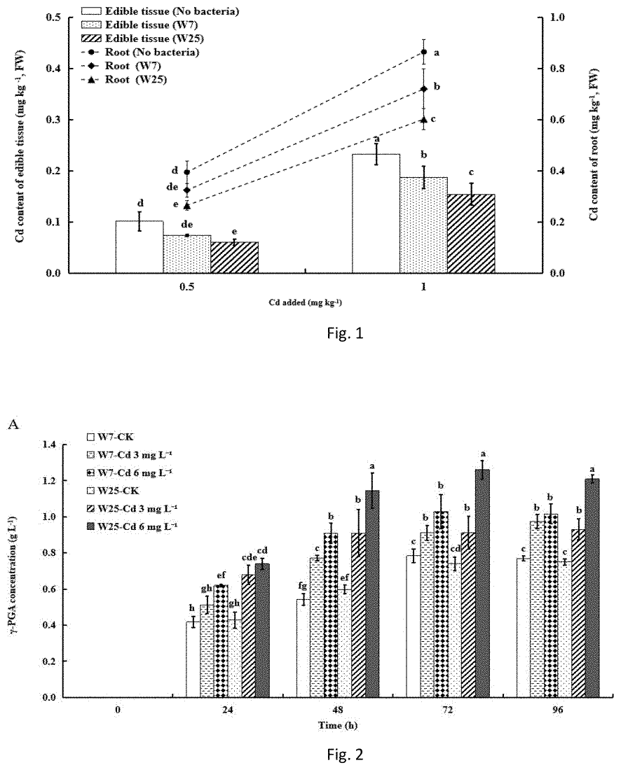

Experimental results are shown in FIG. 1 and Table 1:

TABLE 1

| Cd added | Dry weight | Vc content | Soluble protein |
|---|---|---|---|
| (mg · kg⁻¹) | (g) | (mg · g⁻¹) | content (mg · g⁻¹) |
| 0* | | | |
| No bacteria | 1.73 ± 0.37c | 0.16 ± 0.03cd | 1.45 ± 0.22bc |
| W7 | 2.54 ± 0.23ab | 0.22 ± 0.01ab | 1.58 ± 0.33ab |
| W25 | 3.20 ± 0.07a | 0.22 ± 0.02ab | 1.82 ± 0.03a |
| 0.5* | | | |
| No bacteria | 1.92 ± 0.35bc | 0.15 ± 0.01d | 1.13 ± 0.09c |
| W7 | 2.70 ± 0.13a | 0.21 ± 0.01bc | 1.62 ± 0.13ab |
| W25 | 3.15 ± 0.46a | 0.26 ± 0.05a | 1.62 ± 0.21ab |
| 1* | | | |
| No bacteria | 1.76 ± 0.20c | 0.14 ± 0.01d | 1.13 ± 0.08c |
| W7 | 2.56 ± 0.28ab | 0.18 ± 0.01bcd | 1.33 ± 0.07bc |
| W25 | 2.83 ± 0.39a | 0.21 ± 0.05abc | 1.55 ± 0.22ab |

It can be seen from the above results that the strain W7 reduces the Cd content in the edible tissue of lettuce by 17% to 33%, increases the biomass of the edible tissue of lettuce by 41% to 47%, increases the Vc content in the edible tissue of lettuce by 29% to 40%, and increase the soluble protein content in the edible tissue of lettuce by 9% to 43%.

The strain W25 reduces the Cd content in the edible tissue of lettuce by 30% to 41%, increases the biomass of the edible tissue of lettuce by 61% to 85%, increases the Vc content in the edible tissue of lettuce by 38% to 73%, and increase the soluble protein content in the edible tissue of lettuce by 37% to 43%.

### Example 4

Effects of the Strains W7 and W25 Respectively Inoculated into Soil Filtrates on Water-Soluble Cd and γ-PGA Content

2.5 kg of soil was added into 10 L of deionized water, shaken at 150 rpm for 48 h, and centrifuged at 5,000 rpm for 15 min, a supernate was collected, filtered by using a millipore filter (with a pore diameter of 0.45 m), and sterilized; then, the sterilized supernate was uniformly mixed with a sterile basic fermentation medium according to a volume ratio of 4:1 to prepare a sterile mixture. Mass concentrations of various components of the sterile basic fermentation medium are as follows: 3% glucose, 0.25% yeast extract, 2% glutamate, 0.05% potassium dihydrogen phosphate, 0.05% K₂HPO₄, 0.01% MgSO₄.7H₂O, and the balance of water, and a pH value of the medium is 7.2±0.1. Cd²⁺ (CdCl₂.2.5H₂O) at different concentrations was added into the above prepared sterile mixture to make final Cd2⁺ concentrations be 0, 3 and 6 mg·L⁻¹, respectively; 100 mL of mixture was added into a triangular flask and inoculated with the bacterial suspension prepared in Example 2 according to an inoculation amount of 1% (v:v), 6 parallels were prepared for each concentration group, and the mixture with the final Cd2⁺ concentration of 0 was taken as a control.

The mixture was cultured at 37° C. and 150 rpm, the γ-PGA content, the Cd content, OD₆₀₀ values, and pH values of the culture solution were determined at 0, 24, 48, 96 h, respectively.

The growth of bacteria was monitored by determining OD₆₀₀ values, and pH values were determined by using a pH meter.

A method for determining the γ-PGA content: the γ-PGA content was determined with reference to the method disclosed by Wei Zeng, et al., (2013) (An integrated high-throughput strategy for rapid screening of poly (γ-glutamic acid)-producing bacteria. Appl Microbiol Biotechnol (2013) 97: 2163-2172).

A method for determining the Cd content: the cadmium concentration was determined by using an inductively coupled plasma-optical emission spectrometry (ICP-OES) (Optima 2100DV, Perkin-Elmer) with reference to the method disclosed by Gao Yuanyuan, Peng Zhaofeng, Qiu Haiou, Gu Yansheng, and Cheng Wei in Determination of heavy metal elements in dominant plants from Hubei Zigui Yueliangbao gold mine tailings with ICP-OES [J], Chinese Journal of Analysis Laboratory, 2016, 35(5): 521-525.

Determination methods of the strain W25 were the same as those of the strain W7.

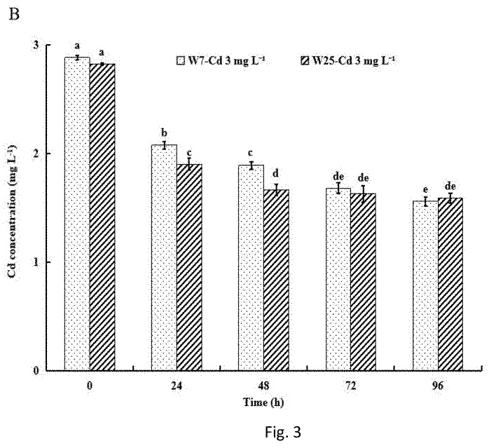

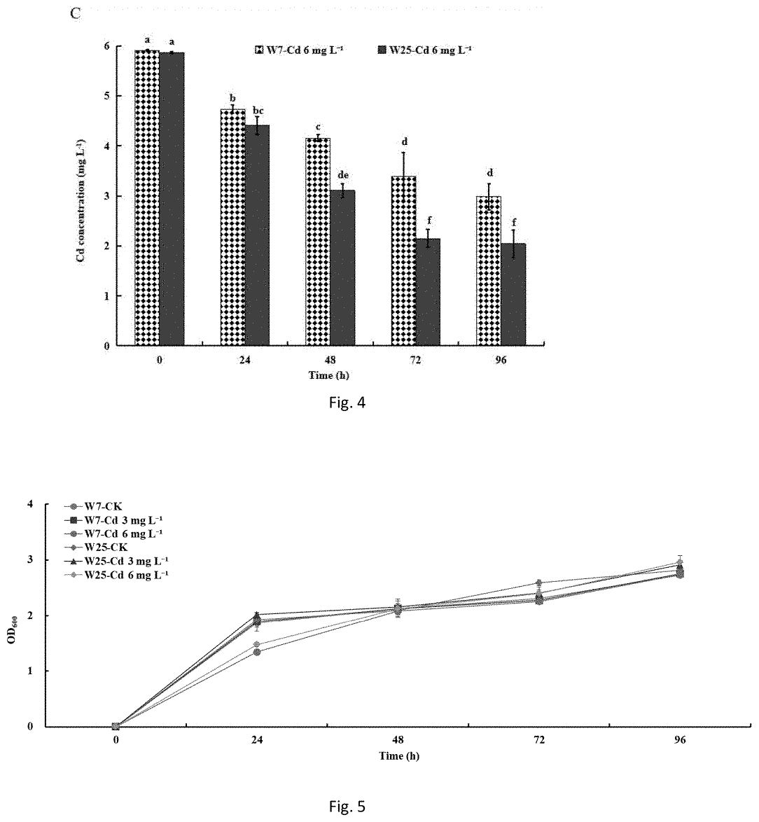

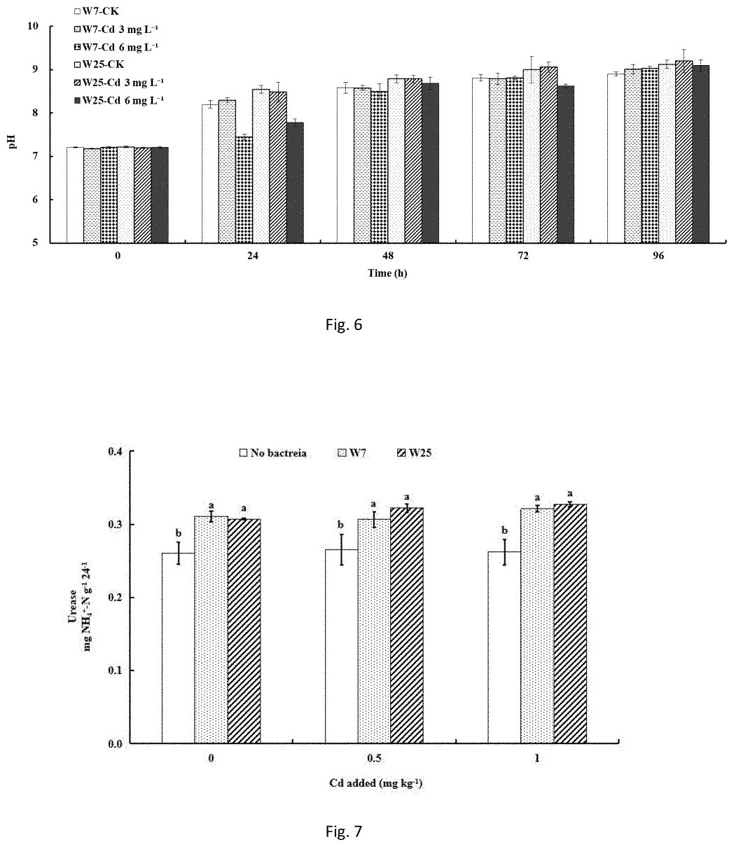

Experimental results are shown in FIG. 2, FIG. 3, FIG. 4, FIG. 5, and FIG. 6.

It can be seen from FIG. 2 that the γ-PGA content changes greatly with the culture time, under the condition of the same culture time, the higher the Cd content in the culture solution, the greater the γ-PGA yield of the strain W7; the same phenomenon occurs in the experiment of the strain W25; and under the conditions of the same culture time and the same Cd content in the culture solution, the γ-PGA yield of the strain W25 is greater than that of the strain W7.

It can be seen from FIG. 3 and FIG. 4 that the Cd content in the culture solution is reduced significantly with the culture time, the columns at the culture time of 96 h in the FIG. 4 show that the Cd content in the culture solution corresponding to the strain W7 is reduced by 50%; and the Cd content in the culture solution corresponding to the strain W25 is reduced by more than 60%.

It can be seen from FIG. 5 that the OD₆₀₀ value of the culture solution is continuously increased with the culture time, the OD₆₀₀ values of various culture solution are different at the culture time of 24 h, and the OD₆₀₀ values of various culture solutions are almost the same at the culture time of 48 h, that is, the bacterial concentrations are basically the same, and it can be seen from the OD₆₀₀ values at 72 h and 96 h that the bacteria concentrations in various culture solutions have little difference and are basically the same at the later culture stage.

It can be seen from FIG. 6 that the pH value of the culture solution is continuously increased with the culture time and barely changes at the later culture stage.

### Example 5

Detection of increase of the activity of enzymes in the soil by the strains W7 and W25, respectively, and detection of increase of relative abundances of Proteobacteria, Firmicutes, Sphingomonas, and Bacillus by the strains W7 and W25, respectively

The soil (rhizosphere soil) closely bound to the root in the pot experiment of Example 3 was collected for the following analysis; and a urease activity in the collected soil (Soil Sci Ch. Acad, 1980; Guan, et al., 1986) was determined.

Genome DNA of bacteria in the collected soil sample was extracted by using a rapid DNA extraction kit (MP Biomedicals, Santa Ana, Calif.), and stored at −20° C. for the following analysis; the quality and quantity of the extracted DNA were respectively determined by using a spectrophotometry (NanoDrop 1000, Thermo Scientific, USA) and by means of gel-electrophoresis. The extracted DNA was amplified by using primers 338F (5′-actcctagggggcagca-3′) and 806R (5′-GGACTACHVGGGTWTCTAAT-3′) to target the V4 region of 16s rRNA of the bacteria. High-throughput sequencing was carried out on Illumina Hiseq 2000 (Illumina Inc., San Diego, USA).

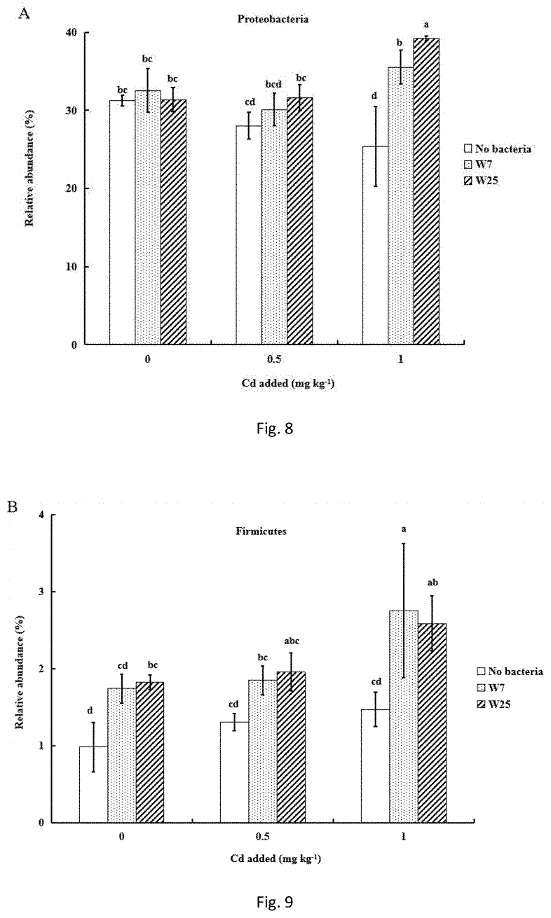

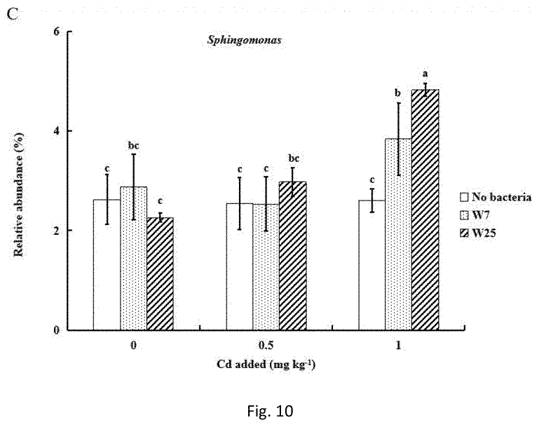

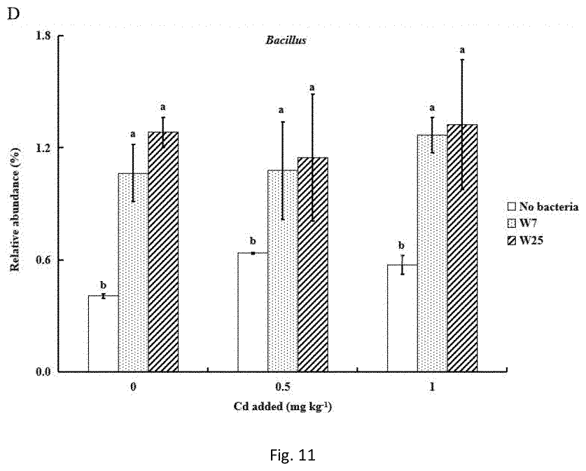

Experimental results are shown in FIG. 7, FIG. 8, FIG. 9, FIG. 10, and FIG. 11.

It can be seen from FIG. 7 that the strains W7 and W25 increase the urease activity in the soil to a certain extent, and when the soil contains Cd, the effects of strains W7 and W25 on increase of the urease activity in the soil are more significant.

It can be seen from FIG. 8 to FIG. 11 that the strains W7 and W25 promote proliferation of Proteobacteria, Firmicutes, Sphingomonas, and Bacillus in the soil to a certain extent, and it can be seen from FIG. 10 that if the Cd content in the soil is relatively high, the promotion effects of the strains W7 and W25 on proliferation of Sphingomonas are more significant, and the promotion effect of the strain W25 on proliferation of Sphingomonas is better than that of the strain W7.

Based on the above, the strains W7 and W25 of the present disclosure can reduce heavy metal contents, especially cadmium (Cd) contents, in plants, promote growth of plants, increase Vc and soluble protein contents in plants, and increase a microbial abundance and a urease activity in the soil at the same time, and can be widely applied to the fields of soil improvement, crop biomass increase, and soil heavy metal restoration.

What is claimed is:

1. A Bacillus strain W7, taxonomically named Bacillus subtilis W7, deposited in China General Microbiological Culture Collection Center (CGMCC) at No. 3, Yard 1, Beichen West Road, Chaoyang District, Beijing, China on Jun. 11, 2020, with the number of CGMCC20043.

2. A culture method of the stain W7 according to claim 1, comprising the following steps:
inoculating the strain W7 onto a solid medium and culturing same at 28 to 32° C. for 2 to 3 d, picking colonies, inoculating same into a liquid medium, and culturing same at 35 to 37° C. and 150 to 200 rpm for 16 to 20 h to obtain a fermentation broth of the strain W7, wherein the solid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, 20 g/L of agar, and the balance of water; and the liquid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, and the balance of water.

3. Application of the strain W7 according to claim 1 in preparation of a biological agent,
wherein, preferably, the biological agent is a liquid agent containing the strain W7 at a concentration of more than 1×10⁸ CFU/mL.

4. Application of the strain W7 according to claim 1 in crop planting,
wherein, preferably, the strain W7 is used to reduce a heavy metal cadmium content in a vegetable;
preferably, the strain W7 is used to increase Vitamin C and soluble protein contents in the vegetable; and
further preferably, the vegetable is lettuce.

5. Application of the strain W7 according to claim 1 in soil improvement,
wherein, preferably, the strain W7 is used to immobiliza the heavy metal cadmium in the soil;
preferably, the strain W7 is used to promote proliferation of microbes in the soil; and
further preferably, the strain W7 is used to promote proliferation of one or more of Proteobacteria, Firmicutes, Sphingomonas, and Aneurinibacillus in the heavy metal cadmium-contaminated soil.

6. A Bacillus strain W25, taxonomically named Bacillus amyloliquefaciens, deposited in China General Microbiological Culture Collection Center (CGMCC) at No. 3, Yard 1, Beichen West Road, Chaoyang District, Beijing, China on Jun. 11, 2020, with the number of CGMCC20042.

7. A culture method of the strain W25 according to claim 6, comprising the following steps:
inoculating the strain W25 onto a solid medium and culturing same at 28 to 32° C. for 2 to 3 d, picking colonies, inoculating same into a liquid medium, and culturing same at 35 to 37° C. and 150 to 200 rpm for 16 to 20 h to obtain a fermentation broth of the strain W25, wherein the solid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, 20 g/L of agar, and the balance of water; and the liquid medium contains 10.0 g/L of sucrose, 0.5 g/L of (NH₄)₂SO₄, 2.0 g/L of Na₂HPO₄, 0.1 g/L of NaCl, 0.191 g/L of KCl, 0.5 g/L of MgSO₄.7H₂O, and the balance of water.

8. Application of the strain W25 according to claim 6 in preparation of a biological agent,
wherein, preferably, the biological agent is a liquid agent containing the strain W25 at a concentration of more than 1×10⁸ CFU/mL.

9. Application of the strain W25 according to claim 6 in crop planting,
wherein, preferably, the strain W25 is used to reduce a heavy metal cadmium content in a vegetable;
preferably, the strain W25 is used to increase Vitamin C and soluble protein contents in the vegetable; and
further preferably, the vegetable is lettuce.

10. Application of the strain W25 according to claim 6 in soil improvement,
wherein, preferably, the strain W25 is used to immobiliza the heavy metal cadmium in the soil;
preferably, the strain W25 is used to promote proliferation of microbes in the soil; and
further preferably, the strain W25 is used to promote proliferation of one or more of Proteobacteria, Firmicutes, Sphingomonas, and Aneurinibacillus in the heavy metal cadmium-contaminated soil.
